(19) United States
(12) Patent Application Publication
(10) Pub. No.: US 2005/0256390 A1
(43) Pub. Date: Nov. 17, 2005

(54) Imaging medical examination apparatus with automated patient positioning

(76) Inventors: Dorothea Laux, Erlangen (DE); Mike Muller, Mohrendorf (DE)

Correspondence Address: SCHIFF HARDIN LLP;Patent Department, 6600 Sears Tower, 233 South Wacker Drive, Chicago, IL, 60606, US

(21) Appl. No.: 11/096,136

(22) Filed: Mar. 31, 2005

(30) Foreign Application Priority Data
Mar. 31, 2004 (DE) 10 2004 015 858.4

(51) Int. Cl. A61B 5/05

(52) U.S. Cl. 600/407

(57) ABSTRACT

An imaging medical examination apparatus such as a magnetic resonance apparatus has an image acquisition device, a patient bed, a control device to control the image acquisition means and to move the patient bed as well as a monitor connected with the control device for image output. An operator can designate on the monitor a desired new patient bed position for implementation of further examinations, in an examination image of the patient shown on the monitor that has been acquired with a first patient bed position with the imaging medical examination apparatus. The control device automatically determines the position data of the defined bed position and controls movement of the bed dependent on the determined position data such that the bed subsequently assumes the desired new position.

### BACKGROUND OF THE INVENTION

1. Field of the Invention

The present invention concerns an imaging medical examination apparatus, such as a magnetic resonance apparatus, of the type having an image acquisition device, a patient bed, a control device for control of the image acquisition device and to move the patient bed, as well as a monitor connected with the control device for image output.

2. Description of the Prior Art

Optimally precise positioning of the patient is necessary to achieve a high quality image in various types of imaging medical examination apparatuses. The positioning ensues initially with the aid of user interfaces that, in the example of a magnetic resonance imaging apparatus, make use of a light-beam localizer at the entrance of the tube of the scanner. The distance between the light-beam localizer and the system center (in which a rough homogeneity of the generated magnetic fields exists and in which the examination region is to be positioned) is known, such that the bed must be shifted smoothly by this known distance when the body region of the patient to he examined is located on the bed. Overview (scout) images must then be obtained and evaluated. In order to subsequently more precisely examine conspicuous points in the body of the patient, further exposures are necessary, for which the bed must then be moved such that the new point to be acquired optimally lies in the magnetic field center. Only at that location is the image quality optimal. For example, in order to avoid having to move the patient out of the tube again, repositioning of the bed ensues by the specification by the operator of an estimated movement path. This creates the risk that the subsequent, newly acquired image does not exhibit the necessary image quality with regard to the desired point in the body of the patient, if the movement path has been inaccurately estimated.

German OS 101 09 219 discloses a positioning device for imaging diagnosis systems in which the patient can be moved from an external preparation position into the diagnosis system. A camera coupled with a monitor and a computer is arranged in the region of the preparation position above and/or next to the patient bed. Via a region selection device, a desired examination region can be selected in the image on the monitor in order to automatically guide the patient on the patient bed into the selected scan position in the imaging diagnosis system.

German OS 102 32 676 concerns a method and a device for positioning of a patient in a medical diagnosis or therapy device, wherein an image acquisition device acquires an image of the patient, reproduces it on a monitor, and an image processor aids in the positioning of the patient. The image acquisition device is different from the treatment unit of the diagnosis and therapy device. Via the image processor, a body region is detected and a scan region is proposed that covers this region.

U.S. Pat. No. 6,269,501 describes a method for automatically positioning a subject on a movable table, wherein scout data obtained before the actual scan are used. From the scout data, edges as well as the center of the subject are determined. Dependent on predetermined thresholds, it is determined whether the table must be moved into a new position. Given movement for magnetic resonance imaging that is specified in German OS 101 50 138, for adherence with SAR limit values, SAR values are calculated from position data before acquisition of the diagnostic data and the position of the patient relative to a transmission antenna is calculated for planned parameters of the diagnostic measurement, and the parameters are modified if necessary until SAR values lie within the limit values. A magnetic resonance pre-measurement is used for this purpose.

### SUMMARY OF THE INVENTION

An object of the present invention is to provide an imaging medical examination apparatus of the type initially described that allows improved positioning of the patient.

The above is achieved in accordance with the present invention by an imaging medical examination apparatus having a user interface, including a monitor, that allows an operator to designate, in a displayed image at the monitor obtained with the patient in a first patient bed position, a second patient bed position for implementing a further examination of the patient. Based on the designation of the second patient bed position on the monitor relative to the first patient bed position, a control device automatically determines position data for moving the patient bed to the second patient bed position.

The operator thus defines the next conspicuous point to be examined in the acquired image of the patient body, such as by clicking with a mouse or selection via a keyboard, whereupon the control device automatically determines the position data of the newly defined bed position. Marking (designation) the position by means of joystick or touchpad is alternatively possible. Since the new point to be examined is directly marked, and subsequently a precise calculation ensues by the control device, errors due to an inaccurate estimation of the movement path are precluded. It is thus neither necessary to move the patient out of the tube, only to then move the patient back in again, nor are unnecessary images of insufficient quality acquired. The examination is more comfortable for the patient since the bed does not have to be shifted by unnecessarily long extents, and the number of the acquired images can be reduced to only those that are necessary for diagnosis (with possibly a scout image preceding the very first diagnostic image). A reduction of the acquired images is particularly important when the imaging medical examination apparatus operates with x-rays, for example in computed tomography. The examination event can be accelerated and the operator who views the examination images on the monitor receives comparable simple images of high quality. The operator can limit the survey of the acquired images to the significant point without having to think about the movement of the bed. If desired by the operator, manual shifting of the bed, for example to slightly adjust the position, naturally can be possible as an alternative or addition.

In an embodiment the control device recognizes the first bed position as a predetermined image point on the displayed image and the new bed position can be recognized as another image point designated on the monitor image by the operator. The control device thereby obtains the information needed to be able to determine the new position data of the defined bed position. The control device thus “knows” the first bed position as an electronic image point with coordinates referenced to the examination image, The real (current) bed position, for example, is defined at the Image center. The same is true for the new bed position (defined by the operator, for example via mouse click) in the form of another arbitrary image point relative to the center image point defining the real position, Both of these positions can be set in relation to one another as electronic image points in the system of the examination image. The designated image point would cause the new bed position to be located, in the image center of the new (yet to be acquired) examination image, meaning the bed is to be shifted such that the selected image point is virtually moved into the predetermined system center or the predetermined position is moved with regard to the image acquisition device.

For this purpose in accordance with the invention the control device determines of the new position data based on the pixel distance between both image points. The pixel distance can be a scalar in a two-dimensional image when only one shift along the bed axis is desired that, for clarify for the operator, likewise can be shown in the monitor image over the data necessary to the control device for calculation, independently of the representation in the examination image. If the desired new bed position does not lie on the previous bed axis, the pixel distance has at least one x-component and one y-component, This means that the patient bed must be moved in two dimensions.

In an embodiment the control device also controls movement of the bed along the longitudinal axis and/or transverse axis dependent on the new position data after the automatically calculation thereof. The control device thus evaluates the new patient bed position specified by the operator, calculates new position data using the new patient bed position and using the first bed position, and finally initiates the corresponding shifting of the bed to the extent necessary. After the shifting of the bed, the point in the body of the patient that is now to be examined has an optimal position with regard to the system center or the image acquisition device (scanner).

In an embodiment the control device also controls movement of the bed vertically relative to the bed plane dependent on the new position data. Together with the movement along the longitudinal or transverse axis, it is then possible to select any point in the body of the patient as a new examination region. Here the selection of the new bed position is then implemented using a number of two-dimensional images or in a three-dimensional (possibly virtually three-dimensional) representation. After the implementation of the new data acquisition at the desired new bed position, the operator can in turn select another desired bed position for implementation of a further examination.

### DESCRIPTION OF THE DRAWINGS

FIG. 1 schematically illustrates an examination image of the type obtained by and used in the inventive imaging medical examination apparatus.

FIG. 2 schematically illustrates the shifting of the patient bed in the inventive imaging medical examination apparatus.

### DESCRIPTION OF THE PREFERRED EMBODIMENTS

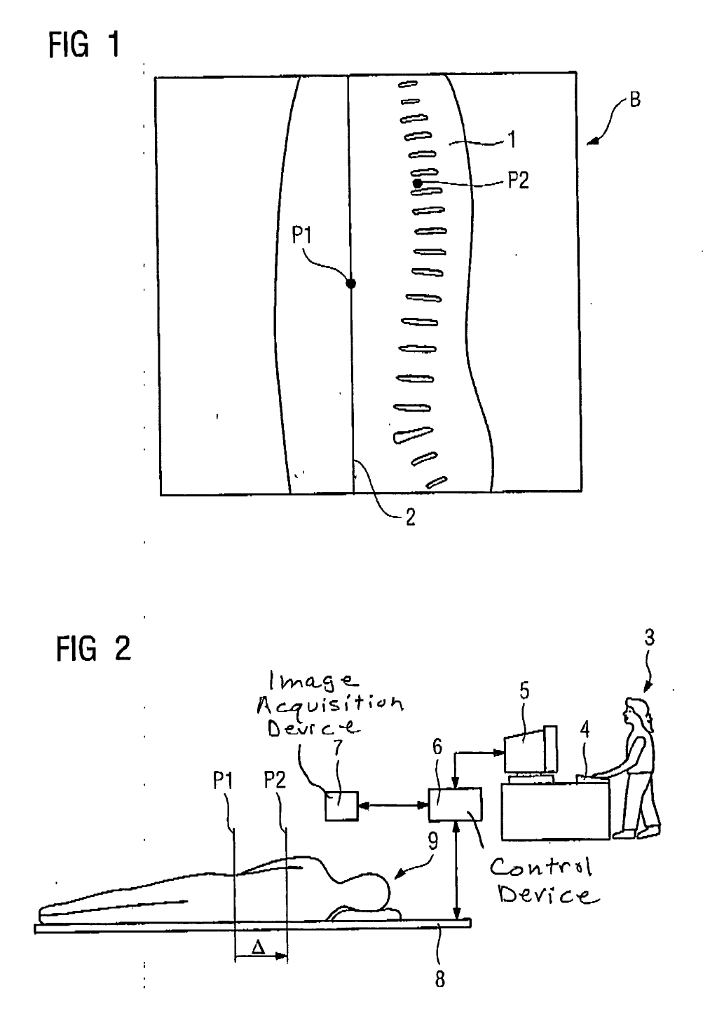

FIG. 1 shows an examination image obtained by and used in the inventive imaging medical examination apparatus. The image of a defined region of the body 1 of the patient. Shown in addition to this is the bed axis 2 on which the first patient bed position P1 lies relative to the image acquisition device, with which the present examination image B has been acquired Located in this bed position P1 is the desired examination region in a specific position with regard to the image acquisition device, in which position a good image acquisition is possible. This position can be, for example, a predetermined position of the bed in the tube of an MR system, in which position the examination region is located in a highly homogenous volume of the basic magnetic field. In the acquisition of the present image B, the patient was lying on his left side in the direction of the longitudinal axis of the bed. Moreover, a new patient bed position P2 is shown that has been selected by an operator, for example via a mouse click. The control device (shown in FIG. 2) then recognizes both of these positions P1 and P2 as image points in the monitor image, whereupon it can determine the new position data. Since, in the shown case, P2 does not lie on the bed axis 2, the bed can be shifted in the longitudinal and transverse directions so that the patient is located in the correct position for acquisition of the new image. P2 is only one position in the examination image that is determined by the anatomy of the patient. Conspicuous items that should be more precisely examined normally are located at this specific point in the patient body 1. In the new examination image, P2 would then occupy the relative position as the current patient bed position, for example in the image center.

FIG. 2 illustrates the principle involved in the shifting of the bed in the inventive imaging medical examination apparatus. An operator 3 controls (via an input device 4, appropriately a keyboard with mouse) software that processes an examination image of the patient shown on the monitor 5. In particular the operator 3 can mark or define a new patient bed position P2 in the examination image. The monitor 5 is connected with a control device 6. The control device 6 controls the image acquisition device 7, which normally is composed of numerous components (not shown here in detail) that are appropriately connected to one another. Moreover, the control device 6 is fashioned for controlling movement of the patient bed 8. The control device 6 receives confirmation data about the successful movement of the bed 8 or the alignment of the image acquisition device 7, whereupon a new positioning of the bed 8 or of the image acquisition device 7 can then ensue automatically or via the operator. The operator 3 marks a new patient bed position P2 in relation to the examination image of the patient 9. The control device 6 then automatically determines new position data based on the pixel distance between both of the bed positions P1 and P2, namely electronic image points associated with the first bed position P1 and the newly defined desired bed position P2. Dependent on these new position data, the control device 6 determines the movement path of the bed 8, which here is specified with Δ, and initiates the corresponding movement of the bed 8. In the example, only a movement along the longitudinal axis of the bed 8 is necessary. Dependent on the level of detail (precision) of the examination image supplied by the image acquisition device 7 or of a digitally processed version thereof in the control device 6, a very precise movement of the bed 8 is thus possible. Given a sufficiently high resolution of the first examination image, qualitatively high-grade exposures can be acquired of the points in the body of the patient 9 that are detected as conspicuous. The control device 6 appropriately includes a microcomputer in which the application software and the algorithms necessary for calculation are stored. In a magnetic resonance apparatus, the patient 9 is surrounded by a tube that is not shown for clarity. Given arrangements that are open in specific spatial directions, a shining of the bed in more than one dimension is possible. The precise determination of the movement path in the inventive examination apparatus saves the patient from the acquisition of unnecessary additional images that were previously necessary due to the merely estimated movement of the bed 8, with the consequence of qualitatively insufficient images.

Although modifications and changes may be suggested by those skilled in the art, it is the intention of the inventors to embody within the patent warranted hereon all changes and modifications as reasonably and properly come within the scope of their contribution to the art.

1. In an imaging medical examination apparatus having a movable patient bed adapted to receive a patient thereon, and an image acquisition device adapted to interact with the patient on said patient bed to obtain an image of at least a part of tire patient, the improvement comprising:
a user interface, including a monitor, allowing an operator to designate, in an image of the patient at the monitor obtained by the image acquisition device with the patient bed in a first patient bed position, a second patient bed position of the patient bed for obtaining a further image of the patient with the image acquisition device, and
a control device for automatically determining position data, from said first patient bed position and said second patient bed position, for use in implementing movement of said patient bed from said first patient bed position to said second patient bed position.

2. An imaging medical examination apparatus as claimed in claim 1 wherein said control device detects said first patient bed position as a first image point in said image displayed at said monitor, and detects said second patient bed position as a second image point in said image displayed at said monitor designated by said operator.

3. An imaging medical examination apparatus as claimed in claim 2 wherein said control device determines said position data based on a pixel distance between said first and second image points.

4. An imaging medical examination apparatus as claimed in claim 1 wherein said control device automatically moves said patient bed along at least one axis selected from the group consisting of a longitudinal axis of said patient bed and a transverse axis of said patient bed, dependent on said position data.

5. An imaging medical examination apparatus as claimed in claim 4 wherein said control device also automatically moves said patient bed vertically dependent on said position data.
